(19) United States
(12) Patent Application Publication
(10) Pub. No.: US 2012/0310088 A1
(43) Pub. Date: Dec. 6, 2012

(54) ULTRASOUND DIAGNOSTIC APPARATUS

(75) Inventors: Masafumi NOGUCHI, Ashigara-kami-gun (JP); Yukiya MIYACHI, Ashigara-kami-gun (JP)

(73) Assignee: FUJIFILM CORPORATION, Tokyo (JP)

(21) Appl. No.: 13/485,023

(22) Filed: May 31, 2012

(30) Foreign Application Priority Data
Jun. 3, 2011 (JP) 2011-125283

(51) Int. Cl. A61B 8/14

(52) U.S. Cl. 600/440

(57) ABSTRACT

An ultrasound diagnostic apparatus adapted to measure the elastic modulus of a vascular wall includes a heartbeat selector for selecting a heartbeat in an M-mode image during the B/M-mode display and, after a heartbeat is selected, displays a B-mode image of a predetermined position in the selected heartbeat. The predetermined position is set in advance during the period of a heartbeat plus two additional periods before and after the heartbeat, with the length of each additional period being 20% on the length of the heartbeat. The ultrasound diagnostic apparatus as such allows the display of an appropriate B-mode image corresponding to the heartbeat to be analyzed.

### BACKGROUND OF THE INVENTION

The present invention relates to an ultrasound diagnostic apparatus which is appropriate for measuring an elastic modulus of a vascular wall, and in particular, to an ultrasound diagnostic apparatus which can display an appropriate B-mode image corresponding to a heartbeat to be analyzed in simultaneous display of a B-mode image and an M-mode image.

An ultrasound diagnostic apparatus using an ultrasound image has hitherto been put into practical use in the field of medicine.

In general, this type of ultrasound diagnostic apparatus has an ultrasound probe (hereinafter, referred to as a probe) and a diagnostic apparatus body. Ultrasonic waves are transmitted from the probe toward a subject, an ultrasonic echo from the subject is received by the probe, and a reception signal is electrically processed by the diagnostic apparatus body to produce an ultrasound image.

Ultrasonic waves are transmitted toward a blood vessel, a cardiac wall, or the like, an ultrasonic echo therefrom is received, and a reception signal is analyzed to obtain the displacement of a cardiac wall, a vascular wall, or the like. The elastic modulus of the vascular wall, the cardiac wall (heart muscle), or the like is measured from the displacement.

For example, JP 10-5226 A describes a technique in which ultrasonic waves are transmitted and received with respect to an object moving in synchronization with the heartbeats (cardiac pulsation) to obtain a reception signal of an ultrasonic echo, the instantaneous position of the object is determined using the amplitude and phase of the reception signal, and the large-amplitude displacement motion of the vascular wall based on the heartbeats is tracked, thereby obtaining the elastic modulus of the blood vessel.

Specifically, the motion velocity waveform of minute vibration of the vascular wall is obtained on the basis of the sequential position of the vascular wall, the tracking trajectory of each of the sections at a predetermined interval in the depth direction in the vascular wall is obtained, and a temporal change in thickness of each section is calculated to obtain the elastic modulus of the blood vessel.

Similarly, JP 2010-233956 A describes an ultrasound diagnostic apparatus which obtains the displacement of a blood vessel or the like from a reception signal of an ultrasonic echo obtained when ultrasonic waves are transmitted and received with respect to an object moving in synchronization with the heartbeats, and obtains an elastic modulus from the displacement.

In this ultrasound diagnostic apparatus, a B-mode image and an M-mode image are produced using a reception signal obtained from an object, such as a blood vessel. Blurring due to hand or body movement is detected from the reception signal of the M-mode image, and the positional variation of the probe and the subject is detected using the reception signal of the M-mode image where the blurring is detected. The accuracy of the reception signal is determined from the detection result, and the displacement of the object is obtained using the reception signal of the M-mode image whose accuracy is determined to be high, and the elastic modulus of the vascular wall or the like is measured from the displacement.

As described in JP 2010-233956 A, in an ultrasound diagnostic apparatus which performs simultaneous display of a B-mode image and an M-mode image, normally, if a freeze button for freezing an image as a still image is depressed, the M-mode image when the freeze button is depressed is frozen, and the B-mode image when the freeze button is depressed is displayed.

For this reason, the B-mode image which is displayed is not necessarily limited to an image appropriate for performing diagnosis or the like.

JP 2004-8350 A describes an ultrasound diagnostic apparatus in which, when simultaneously displaying a B-mode image and an M-mode image, a waveform chart, such as an electrocardiogram, based on activity information of a living body tissue is displayed, a specific time phase of the waveform chart is displayed in the M-mode image, and a B-mode thumbnail image corresponding to the specific time phase is displayed. In the ultrasound diagnostic apparatus described in JP 2004-8350 A, the time phase is arbitrarily selected in the M-mode image, such that a B-mode thumbnail image corresponding to the time phase is displayed.

In the ultrasound diagnostic apparatus described in JP 2004-8350 A, with the above configuration, it is possible to efficiently acquire information relating to the properties of the living body tissue, such as the elastic modulus, or strain, thereby improving efficiency of ultrasound diagnosis.

### SUMMARY OF THE INVENTION

In order to accurately measure the elastic modulus or the like of the vascular wall from the displacement of an object which moves in synchronization with the heartbeats, it is necessary that a heartbeat appropriate for analysis is selected from an M-mode image and a B-mode image is a B-mode image which corresponds to the selected heartbeat, that is, a heartbeat to be analyzed and is also appropriate for analysis.

However, in the conventional ultrasound diagnostic apparatus, it is not always true that an arbitrary heartbeat can be selected from an M-mode image, and even when an arbitrary heartbeat can be selected in an M-mode image, it is not always true that a B-mode image which corresponds to the selected heartbeat and is also appropriate for analysis is displayed.

The invention has been accomplished in order to solve the problems with the prior art, and an object of the invention is to provide an ultrasound diagnostic apparatus capable of displaying a B-mode image and an M-mode image to allow selection of a heartbeat which is appropriate for analysis when measuring a blood vessel elastic modulus from the M-mode image, and displaying a B-mode image which corresponds to the heartbeat selected in the M-mode image and is also appropriate for analysis.

In order to achieve the above object, the present invention provides an ultrasound diagnostic apparatus comprising: an ultrasound probe which has ultrasound transducers transmitting ultrasonic waves, receiving an ultrasonic echo reflected by a subject, and outputting a reception signal according to the ultrasonic echo received; an image producer which produces a B-mode image and an M-mode image from the reception signal output from the ultrasound transducers; a display unit; a display processor which displays at least one of the B-mode image and the M-mode image produced by the image producer on the display unit; a freezer which freezes a display image to a still image in a state where the B-mode image and the M-mode image are displayed; and a heartbeat selector which selects a heartbeat in the M-mode image in a state where the B-mode image and the M-mode image are frozen as still images, wherein, after the heartbeat is selected in the M-mode image, in correspondence to a predetermined position set in advance within a period from an early 20% of the heartbeat to a late 20% of the heartbeat relative to a length of the heartbeat, the display processor displays a B-mode image of the predetermined position in the selected heartbeat on the display unit.

It is preferable that the ultrasound diagnostic apparatus as above further comprises a heartbeat detector which detects heartbeats in the M-mode image, and, after the heartbeat detector detects the heartbeats, the display processor displays all the heartbeats detected by the heartbeat detector in the M-mode image.

The heartbeat detector preferably analyzes the M-mode image to detect the heartbeats in the M-mode image.

It is preferable that the ultrasound diagnostic apparatus further comprises a selector which selects the predetermined position.

It is preferable that the heartbeat selector decides that a latest heartbeat wholly acquired from start to end in an M-mode image is selected among the heartbeats detected by the heartbeat detector.

Preferably, the predetermined position is set outside a period after a heart diastole and before a heart systole.

It is preferable that the ultrasound diagnostic apparatus further comprises a position adjuster which adjusts a position of the heartbeat selected by the heartbeat selector.

It is preferable that, after the position of the heartbeat is adjusted by the position adjuster, the display processor changes the B-mode image to be displayed on the display unit to an image of a corresponding position in accordance with heartbeat position adjustment and the predetermined position.

It is preferable that the ultrasound diagnostic apparatus further comprises a vascular wall boundary setter which sets a position of a vascular wall boundary in the B-mode image.

It is preferable that the ultrasound diagnostic apparatus further comprises a region-of-interest setter which sets a region of interest in the B-mode image displayed on the display unit.

Preferably, a frame rate of ultrasonic waves by the ultrasound transducers is increased in response to an instruction to set the region of interest to be higher than before the instruction to set the region of interest.

In the ultrasound diagnostic apparatus of the invention configured as above, when a B-mode image and an M-mode image are displayed simultaneously to perform measurement of blood vessel elasticity or the like, a heartbeat which is appropriate for analysis for performing an intended measurement can be selected from the M-mode image, and in correspondence to a predetermined position set in advance within a predetermined period corresponding to the heartbeat, a B-mode image at the predetermined position in the selected heartbeat is displayed.

For this reason, in the ultrasound diagnostic apparatus of the invention, a heartbeat which is appropriate for the measurement of the blood vessel elastic modulus or the like is selected, and a B-mode image which corresponds to the selected heartbeat and is appropriate for analysis can be then displayed, thereby stably performing accurate measurement of the blood vessel elastic modulus or the like.

### BRIEF DESCRIPTION OF THE DRAWINGS

FIG. 1 is a diagram conceptually illustrating an example of an ultrasound diagnostic apparatus of the invention.

FIG. 2 is a block diagram conceptually illustrating the configuration of the ultrasound diagnostic apparatus illustrated in FIG. 1.

FIG. 3 is a flowchart for explaining an example of elasticity measurement of a vascular wall in the ultrasound diagnostic apparatus illustrated in FIG. 1.

FIG. 4 is a conceptual diagram for explaining an ultrasound diagnosis for elasticity measurement of a vascular wall.

FIGS. 5A and 5B are conceptual diagrams illustrating an example of image display in the ultrasound diagnostic apparatus illustrated in FIG. 1.

FIGS. 6A and 6B are conceptual diagrams illustrating an example of image display in the ultrasound diagnostic apparatus illustrated in FIG. 1, and FIG. 6C is a conceptual diagram for explaining the action of an ultrasound diagnostic apparatus of the invention.

FIGS. 7A to 7C are conceptual diagrams illustrating an example of image display in the ultrasound diagnostic apparatus illustrated in FIG. 1.

FIGS. 8A and 8B are conceptual diagrams illustrating an example of image display in the ultrasound diagnostic apparatus illustrated in FIG. 1.

FIG. 9 is a conceptual diagram illustrating an example of image display in the ultrasound diagnostic apparatus illustrated in FIG. 1.

FIGS. 10A to 10G are conceptual diagrams illustrating an example of image display in the ultrasound diagnostic apparatus illustrated in FIG. 1.

FIGS. 11A and 11B are conceptual diagrams illustrating an example of image display in the ultrasound diagnostic apparatus illustrated in FIG. 1.

FIG. 12 is a conceptual diagram illustrating an example of image display in the ultrasound diagnostic apparatus illustrated in FIG. 1.

### DETAILED DESCRIPTION OF THE INVENTION

Hereinafter, an ultrasound diagnostic apparatus of the invention will be described in detail on the basis of a preferred example illustrated in the accompanying drawings.

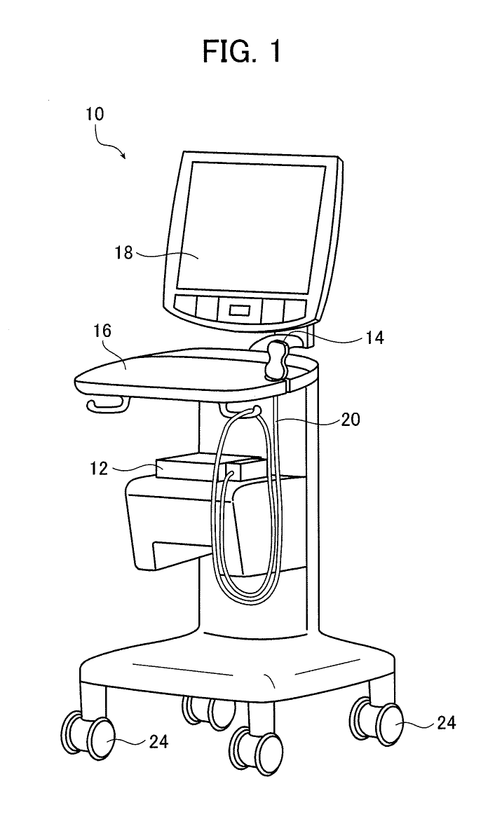

FIG. 1 conceptually illustrates the appearance of an example of the ultrasound diagnostic apparatus of the invention.

As illustrated in FIG. 1, an ultrasound diagnostic apparatus 10 basically has a diagnostic apparatus body 12, an ultrasound probe 14, an operating panel 16, and a display 18. Casters 24 are arranged at the lower end of the ultrasound diagnostic apparatus 10, such that the apparatus can be easily moved by human power.

The ultrasound probe 14 (hereinafter, referred to as a probe 14) performs transmission/reception of ultrasonic waves, and supplies a reception signal according to a received ultrasonic echo to the diagnostic apparatus body 12.

The probe 14 is a known ultrasound probe which is used in various ultrasound diagnostic apparatuses. The probe 14 has so-called ultrasound transducers (ultrasonic piezoelectric transducers) arranged in a one-dimensional or two-dimensional array which transmit ultrasonic waves toward a subject, receive an ultrasonic echo reflected by the subject, and output an electrical signal (reception signal) according to the received ultrasonic echo.

In the invention, the type of the probe 14 is not particularly limited, and various types, such as a convex type, a linear type, and a sector type, may be used. An external probe or a probe for an ultrasound endoscope, such as a radial scan type, may be used. The probe 14 may have ultrasound transducers for receiving second and higher harmonics of transmitted ultrasonic waves which correspond to harmonic imaging.

In the illustrated example, the probe 14 and the diagnostic apparatus body 12 are connected together by a cable 20. However, the invention is not limited thereto, a transmission circuit 28, a reception circuit 30, a transmission/reception controller 32, and the like described below may be arranged in the probe 14, and the probe 14 and the diagnostic apparatus body 12 may be connected together by wireless communication.

The display 18 is a known display (display device).

In the ultrasound diagnostic apparatus 10, as in various ultrasound diagnostic apparatuses, the display 18 displays an ultrasound image according to a reception signal output from the probe 14, information of the subject, selection means or instruction means for operation by a GUI (Graphical User Interface), a region of interest (hereinafter, referred to as ROI), an elasticity measurement result of a vascular wall described below, and the like.

The operating panel 16 is provided to operate the ultrasound diagnostic apparatus 10.

Though not illustrated, in the ultrasound diagnostic apparatus 10, the operating panel 16 has arranged therein selection means for selecting various modes, such as a B mode and an M mode, a trackball (track pad/touch pad) for moving a cursor, a line, or the like displayed on the display 18, a set button for determining (confirming) selection or operation, a freeze button for switching between motion image display and still image display, changing means for changing the visual field depth of an ultrasound image, gain adjusting means, a zoom button for enlarging an ultrasound image, and the like.

As the modes of the ultrasound diagnostic apparatus 10, in addition to the modes of the normal ultrasound diagnostic apparatus, such as a B mode and an M mode, a VE mode (Vascular Elasticity Mode) for measuring the elastic modulus of the vascular wall is set.

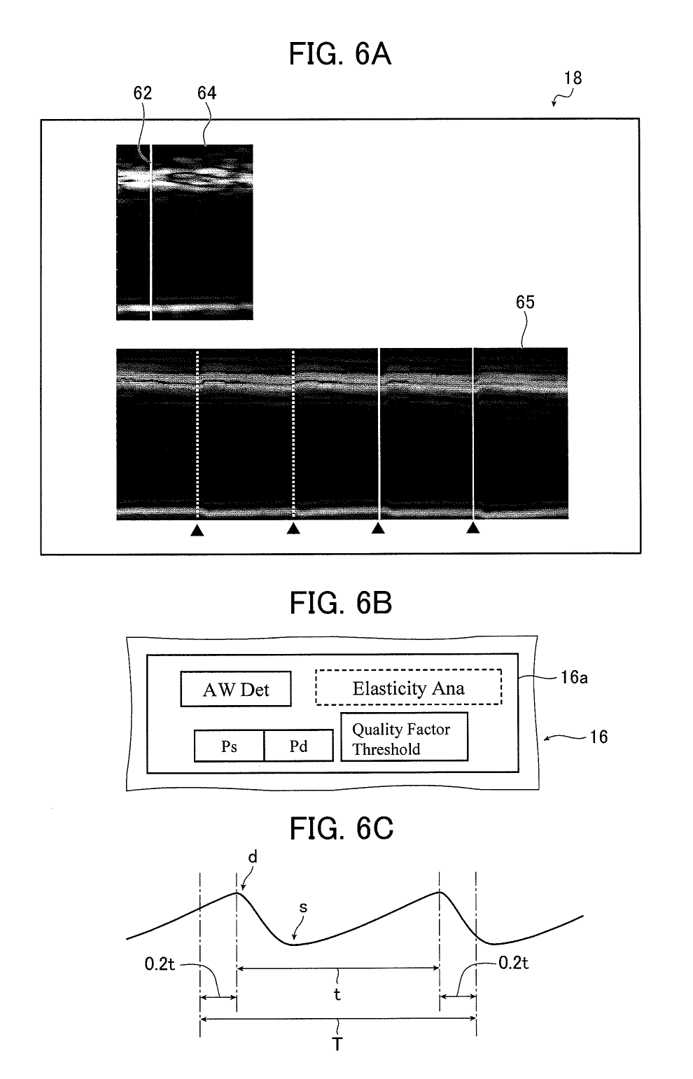

Though not illustrated, the operating panel 16 also has arranged therein a touch panel 16a (see FIG. 6B) which is a display device for operation by GUI.

The diagnostic apparatus body 12 controls the overall operation of the ultrasound diagnostic apparatus 10, and also performs various processes for producing an ultrasound image according to the reception signal output from the probe 14, displaying the ultrasound image on the display 18, and measuring a blood vessel elastic modulus.

The diagnostic apparatus body 12 is constituted using, for example, a computer.

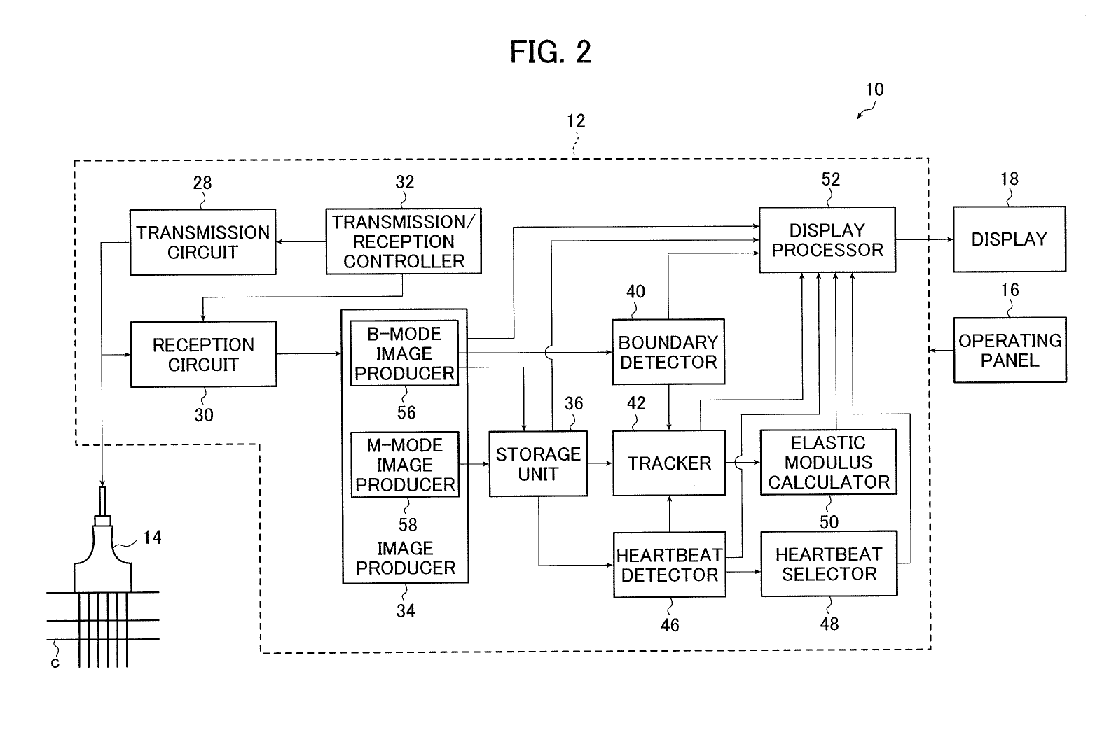

FIG. 2 is a block diagram conceptually illustrating the configuration of the ultrasound diagnostic apparatus 10.

As illustrated in FIG. 2, the diagnostic apparatus body 12 has a transmission circuit 28, a reception circuit 30, a transmission/reception controller 32, an image producer 34, a storage unit 36, a boundary detector 40, a tracker 42, a heartbeat detector 46, a heartbeat selector 48, an elastic modulus calculator 50, and a display processor 52.

The image producer 34 has a B-mode image producer 56 and an M-mode image producer 58.

The above-mentioned probe 14 is connected to the transmission circuit 28 and the reception circuit 30. The transmission/reception controller 32 is connected to the transmission circuit 28 and the reception circuit 30. The reception circuit 30 is connected to the image producer 34.

The image producer 34 is connected to the display processor 52. The B-mode image producer 56 and the M-mode image producer 58 of the image producer 34 are connected to the storage unit 36. The B-mode image producer 58 is also connected to the boundary detector 40.

The storage unit 36 is connected to the tracker 42, the heartbeat detector 46, and the display processor 52. The heartbeat detector 46 and the boundary detector 40 are connected to the tracker 42 and the display processor 52 together. The tracker 42 is also connected to the elastic modulus calculator 50, and the elastic modulus calculator 50 is connected to the display processor 52. The heartbeat detector 46 is also connected to the heartbeat selector 48, and the heartbeat selector 48 is also connected to the display processor 52.

The transmission/reception controller 32 sequentially sets the transmission direction of an ultrasonic beam and the reception direction of an ultrasonic echo of the probe 14 through the transmission circuit 28 and the reception circuit 30.

The transmission/reception controller 32 also has a transmission control function of selecting a transmission delay pattern in accordance with the set transmission direction and a reception control function of selecting a reception delay pattern in accordance with the set reception direction.

The transmission delay pattern is the pattern of a delay time which is given to an actuation signal of each ultrasound transducer so as to produce an ultrasonic beam to a desired direction by ultrasonic waves transmitted from a plurality of ultrasound transducers of the probe 14. The reception delay pattern is the pattern of a delay time which is given to a reception signal so as to extract an ultrasonic echo from a desired direction by ultrasonic waves received by a plurality of ultrasound transducers.

A plurality of transmission delay patterns and a plurality of reception delay patterns are stored in an internal memory (not illustrated), and are appropriately selected and used depending on the situation.

The transmission circuit 28 includes a plurality of channels, and produces a plurality of actuation signals which are respectively applied to a plurality of ultrasound transducers of the probe 14. At this time, it is possible to give the delay time to each of a plurality of actuation signals on the basis of the transmission delay pattern selected by the transmission/reception controller 32.

The transmission circuit 28 may adjust the delay amount of each of a plurality of actuation signals such that ultrasonic waves transmitted from a plurality of ultrasound transducers of the probe 14 produce an ultrasonic beam, and may respectively supply the adjusted actuation signals to the ultrasound transducers. Alternatively, the transmission circuit 28 may supply to the probe 14 a plurality of actuation signals made up such that ultrasonic waves transmitted from a plurality of ultrasound transducers at a time cover the entire imaging region of the subject.

Similarly to the transmission circuit 28, the reception circuit 30 includes a plurality of channels. The reception circuit 30 amplifies a plurality of analog signals received through a plurality of ultrasound transducers and converts the amplified analog signals to digital reception signals.

A reception focus process is performed by giving the delay time to each of a plurality of reception signals on the basis of the reception delay pattern selected by the transmission/reception controller 32 and adding the reception signals. With this reception focusing process, the focus of the ultrasonic echo is narrowed to produce a sound ray signal (sound ray data).

The produced sound ray data is supplied to the image producer 34.

The image producer 34 performs a preprocess, such as Log (logarithmic) compression or gain adjustment, on the supplied sound ray data to produce image data of the ultrasound image, converts (raster-converts) the image data to image data based on a normal television signal scan system, performs a necessary image process, such as a gradation process, on the image data and outputs the image data to the display processor 52.

The image producer 34 has a B-mode image producer 56 which produces a B-mode image, and an M-mode image producer 58 which produces an M-mode image. The B-mode image and the M-mode image may be produced by a known method.

The display processor 52 produces display data for display on the display 18 in accordance with image data of the ultrasound image supplied from the image producer 34, image data of the ultrasound image read from the storage unit 36, operation (input instruction) on the operating panel 16, the heartbeat detection result or heartbeat selection described below, the measurement result (analysis result) of a vascular wall elastic modulus described below, and the like, and displays the display data on the display 18.

In the ultrasound diagnostic apparatus 10 of the illustrated example, the storage unit 36, the boundary detector 40, the tracker 42, the heartbeat detector 46, the heartbeat selector 48, and the elastic modulus calculator 50 of the diagnostic apparatus body 12 are primarily used in the VE mode in which the elastic modulus of the vascular wall is measured.

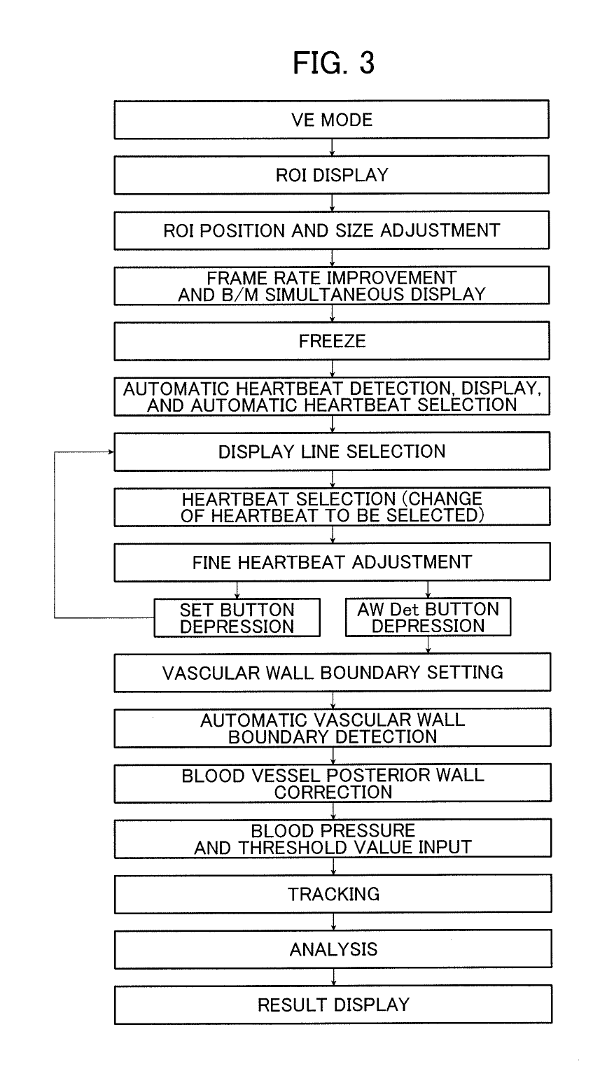

Hereinafter, the respective units, such as the storage unit 36 and the boundary detector 40, and the ultrasound diagnostic apparatus 10 of the invention will be described in detail by describing the action of the ultrasound diagnostic apparatus 10 in the VE mode with reference to a flowchart of FIG. 3 and FIGS. 5 to 12.

In the following description, with regards to the display of the display 18, the display processor 52 performs necessary process, such as line production, even though not particularly described.

If an ultrasound diagnosis by the ultrasound diagnostic apparatus 10 starts, under the control of the transmission/reception controller 32, the transmission circuit 28 causes the ultrasound transducers of the probe 14 to transmit ultrasonic waves, and the reception circuit 30 processes the reception signal output from the probe 14 to produce a sound ray signal and outputs the sound ray signal to the image producer 34.

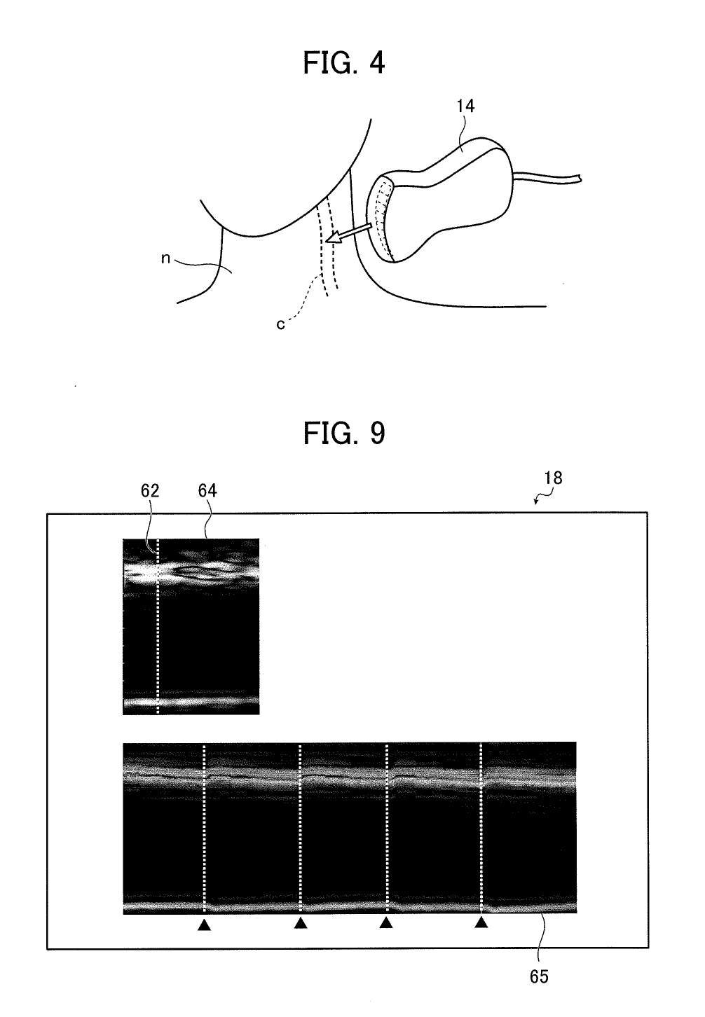

As an example, the B mode is selected, as conceptually illustrated in FIG. 4, a carotid artery c of the subject is used as a measurement target, and the probe 14 is brought into contact with a neck n. In this case, a B-mode image produced by the image producer 34 (B-mode image producer 56) is processed by the display processor 52 and displayed on the display 18.

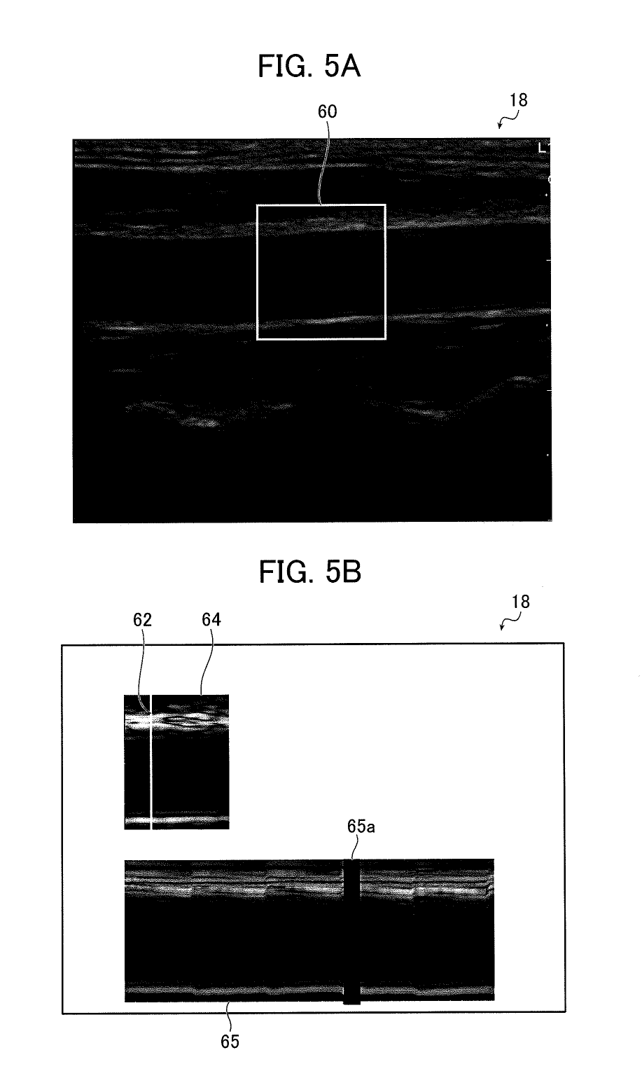

If the intended carotid artery c can be observed appropriately, and the VE mode is selected by mode selection means of the operating panel 16 (in the following description, “of the operating panel 16” is omitted), as conceptually illustrated in FIG. 5A, the display processor 52 displays the ROI 60 representing the region of interest in the B-mode image.

In this state, the position of the ROI 60 in the B-mode image can be moved by operation of the trackball. If the set button is depressed, the position of the ROI 60 is fixed, and the size of the ROI 60 can be changed by operation of the trackball.

Each time the set button is depressed, the position change of the ROI 60 and the size adjustment of the ROI 60 can be alternately carried out.

If the zoom button is depressed in this state, the adjustment of the position or the size of the ROI 60 ends, and an instruction to set the ROI 60 is made. In response to this situation, the transmission/reception controller 32 increases the frame rate to be higher than before the instruction to set the ROI 60 (for example, to be equal to or higher than 200 Hz, or at least five times higher than before the ROI setting instruction). If the zoom button is depressed, the M-mode image producer 58 starts to produce an M-mode image of the ROI 60, and as illustrated in FIG. 5B, a B-mode image 64 where the portion of the ROI 60 is enlarged and an M-mode image 65 of (a selection line 62 of) the ROI 60 are displayed simultaneously.

The simultaneous display (dual mode display) of the B-mode image 64 and the M-mode image 65 may be performed in the same manner as so-called B/M-mode display in the known ultrasound diagnostic apparatus.

In FIG. 5B, the upper side is the B-mode image 64, and the lower side is the M-mode image 65.

In the B-mode image 64, the horizontal direction of the drawing is the azimuth direction (the arrangement direction of the ultrasound transducers (in the two-dimensional arrangement, the longitudinal direction)), and the vertical direction is the depth direction (the transmission/reception direction of ultrasonic waves). The upper side in the depth direction is the side on which the depth is shallow (the probe 14 side).

The selection line 62 which extends in the depth direction to select the display position of the M-mode image (the display line of the M-mode image) in the azimuth direction in the B-mode image is displayed in the B-mode image. The selection line 62 is movable in the azimuth direction (the left-right direction in the drawing) by the trackball.

In the M-mode image 65, the horizontal direction is the direction of the time axis. The time flows from left to right, and the left side of a gap 65a becomes a current frame (that is, the right side of the gap 65a is a previous frame). Similarly to the B-mode image 64, the vertical direction is the depth direction. The upper side in the depth direction is the side on which the depth is shallow.

In FIG. 5B, the M-mode image 65 displayed on the display 18 is the M-mode image 65 at the position of the selection line 62 set in advance.

The M-mode image producer 58 produces an M-mode image at a predetermined position (a predetermined position set in advance or a selected position) in the azimuth direction or a selected position in the azimuth direction as well as over the entire region of the B-mode image 64 in the azimuth direction.

The B-mode image (B-mode image data) of the ROI 60 produced by the B-mode image producer 56 and the M-mode image (M-mode image data) produced by the M-mode image producer 58 are stored in the storage unit 36 together.

The temporal amount of an image which is stored in the storage unit 36 is not particularly limited, while a duration including two or more common heartbeats is preferred. Accordingly, it is preferable that the storage unit 36 stores the latest B-mode image and M-mode image which are each three seconds or longer in duration.

As described above, the selection line 62 can be moved in the azimuth direction by the trackball.

The position of the selection line 62 and the M-mode image are moved together. That is, if the selection line 62 is moved in the left-right direction by the trackball, the display processor 52 displays the M-mode image of the position of the selection line 62 on the display 18.

The operator depresses the freeze button if it is determined that an appropriate image is obtained.

If the freeze button is depressed, the display processor 52 reads necessary image data from the storage unit 36, and as illustrated in FIG. 6A, the display processor 52 rearranges the M-mode image 65 of the position of the selection line 62 such that the time at which the freeze button is depressed is on the rightmost side and displays the still image of the M-mode image 65 with the still image of the B-mode image 64 of a predetermined position described below on the display 18. Simultaneously, the selection line 62 becomes a broken line and is not movable (inactive state).

As illustrated in FIG. 6B, an “AW Det” button for instructing to set the boundary of the vascular wall described below, an “Elasticity Ana” button for instructing to start the analysis of the vascular wall elastic modulus, a “Ps” button and a “Pd” button for inputting the blood pressure of the subject, and a “Quality Factor Threshold” button for inputting a reliability threshold value are displayed on the touch panel 16a of the operating panel 16. At this time, the “Elasticity Ana” button is not selectable.

If the freeze button is depressed, the heartbeat detector 46 detects the heartbeats (automatically detects the heartbeats) for all the M-mode images stored in the storage unit 36. The detection result of the heartbeats is sent to the storage unit 36, and added to the corresponding M-mode image as information.

The detection result of the heartbeats is also sent to the display processor 52 and the heartbeat selector 48.

The method of detecting the heartbeats is not particularly limited. As an example, an M-mode image may be analyzed (for example, tracked in the time direction), and the heartbeats may be detected using the moving velocity (the time at which the velocity starts to increase) in the depth direction of a white line (bright line) extending in the horizontal direction, the pulsation of the motion in the depth direction of the same white line, or the like. Alternatively, an electrocardiograph (electrocardiogram) may be used to detect the heartbeats.

Instead of automatically detecting the heartbeats, the operator may input the position of a heartbeat (the start and end positions of a heartbeat) while viewing the M-mode image.

As illustrated in FIG. 6A, the display processor 52 displays the heartbeat detection result (the start/end position of a heartbeat) in the M-mode image 65 by a triangular mark and a line.

When there is the heartbeat which is not detected, the heartbeat may be displayed at an appropriate position in accordance with the interval of heartbeats prior to and subsequent to the heartbeat in question, or the like.

The heartbeat selector 48 decides that the latest heartbeat among complete heartbeats including from the start to the end thereof (fully imaged heartbeats) is selected in accordance with the heartbeat detection result by the heartbeat detector 46 and sends information to the display processor 52. That is, the heartbeat selector 48 decides that a complete heartbeat which is interposed between two lines, that is, fully imaged, and also which is a heartbeat immediately before the freeze button is depressed is selected and sends information to the display processor 52.

The heartbeat selector 48 attaches information that the same heartbeat is selected to all the M-mode images stored in the storage unit 36.

As an example, the display processor 52 represents the selected heartbeat in the M-mode image 65 by a solid line and represents other heartbeats by a broken line in accordance with the heartbeat selection result by the heartbeat selector 48. In the example illustrated in FIG. 6A, since three complete heartbeats are detected, as described above, the rightmost heartbeat which is the latest complete heartbeat is selected, the start position and the end position (the start time and the end time) of the selected heartbeat are represented by a solid line, and the start/end positions of other heartbeats are represented by a broken line.

The selection and deselection may be distinguished by changing the line color instead of or in addition to the line type.

In the ultrasound diagnostic apparatus 10, thereafter, the operator can select a heartbeat (change the heartbeat to be selected), and the heartbeat confirmed in this step is provided for subsequent analysis for blood vessel elasticity measurement as the finally selected heartbeat. Accordingly, the automatic selection of the heartbeat by the heartbeat selector 48 is, so to speak, provisional heartbeat selection.

The heartbeat which is automatically selected by the heartbeat selector 48 is not limited to the latest complete heartbeat, and may be the prior heartbeat or the oldest detected heartbeat. The ultrasound diagnostic apparatus 10 may have selection means for allowing the operator to select the heartbeat which is automatically selected by the heartbeat selector 48. The selection means for selecting the heartbeat which is automatically selected by the heartbeat selector 48 may be constituted by a known method using a GUI or the like.

If the freeze button is depressed, the B-mode image 64 becomes an image at a predetermined position (the time of the predetermined position (predetermined time phase)) set in advance in the heartbeat selected in the M-mode image 65.

Specifically, in the ultrasound diagnostic apparatus 10, a predetermined position (so to speak, a predetermined position on the time axis of a heartbeat (M-mode image)) where the B-mode image 64 is displayed is set in advance within a period from the time corresponding to the early 20% of the heartbeat to the time corresponding to the late 20% of the heartbeat relative to the length (one beat time) of the heartbeat. That is, as illustrated in FIG. 6C conceptually and imitatively of an M-mode image of the blood vessel posterior wall boundary, if the length of the heartbeat is t, a predetermined position, such as the heartbeat start position or the center of a heartbeat, is set in advance during a period T from the time 0.2 t before the heartbeat to the time 0.2 t after the heartbeat (period of 1.4 t around the heartbeat having the length t).

In FIG. 6C, similarly to FIG. 6A or the like, the progression direction of time is from left to right, the upper side is shallow in depth, and the lower side is deep in depth.

Accordingly, in the ultrasound diagnostic apparatus 10, after the ROI is set, the freeze button is depressed, and heartbeat detection and selection are performed, a B-mode image of a predetermined position set in advance, such as the heartbeat start position or the heartbeat center position, in the selected heartbeat (a B-mode image captured at the time of the predetermined position) is displayed.

In the illustrated example, as an example, the heartbeat start position is set as the predetermined position.

Accordingly, at this time, the display processor 52 reads a B-mode image at the start position of the latest heartbeat automatically selected by the heartbeat selector 48 (a B-mode image captured at the start time of the automatically selected heartbeat) from the storage unit 36, and displays the B-mode image on the display along with the M-mode image 65.

As described above, in simultaneous display of a B-mode image and an M-mode image in a normal ultrasound diagnostic apparatus, a B-mode image which is displayed when the freeze button is depressed is an image at the time when the freeze button is depressed.

Meanwhile, in the ultrasound diagnostic apparatus, when the measurement using variation of a target region corresponding to the heartbeats is performed, for example, when the blood vessel elastic modulus is measured from the displacement of the vascular wall, in order to perform accurate measurement, it is necessary to select a heartbeat appropriate for analysis from the M-mode image and to display a B-mode image corresponding to the selected heartbeat to perform analysis.

However, in the conventional apparatus which displays a B-mode image at the freeze time, a B-mode image corresponding to a heartbeat to be used for analysis is not always displayed. As described in JP 2004-8350 A, an apparatus which displays a B-mode image in a specific time phase by an electrocardiogram or the like is also suggested, but it is not always true that the B-mode image corresponds to the heartbeat selected in the M-mode image.

In contrast, in the ultrasound diagnostic apparatus 10 of the invention, a heartbeat is selected in the M-mode image, and in correspondence to a predetermined position set appropriately during a period from the time corresponding to the early 20% of the heartbeat to the time corresponding to the late 20% of the heartbeat (hereinafter, simply referred to as “period from the early 20% of the heartbeat to the late 20% of the heartbeat”) relative to the length of the heartbeat, a B-mode image of the predetermined position of the selected heartbeat is displayed.

For this reason, according to the invention, the B-mode image corresponding to the heartbeat selected in the M-mode image is displayed, thereby performing suitable analysis or diagnosis. For example, as described below, when the vascular wall boundary is set using a B-mode image, and analysis, such as tracking of the vascular wall in the M-mode image, is performed with the time phase of the B-mode image and the set vascular wall boundary as a start point, accurate tracking becomes possible without performing tracking for extra time. A B-mode image suitable for analysis, such as a B-mode image corresponding to the heart diastole or the heart systole in the selected heartbeat, may be displayed.

In the invention, the B-mode image 64 to be displayed may be excessively separated temporally from the heartbeat selected in the M-mode image 65 before the time corresponding to the early 20% of the selected heartbeat and after the time corresponding to the late 20% of the heartbeat.

As a result, there are cases where the state of the displayed B-mode image 64 is significantly different from the selected heartbeat, making it difficult to perform appropriate analysis or diagnosis. For example, as described below, when tracking is performed in the M-mode image with the position of the B-mode image 64 as a temporal start position, the distance (time) from the start position of the tracking until the selected heartbeat is reached is excessively extended. As a result, tracking errors are likely to occur, and there is a problem in that unwanted information, such as noise due to speckle, is collected in the tracking result.

During the period from the early 20% of the heartbeat to the late 20% of the heartbeat, as the predetermined position where the B-mode image 64 is displayed, various positions, such as the position of the early 10% of the heartbeat (before the start of the heartbeat), the heartbeat start position, the position corresponding to the heart systole, that is, the maximum blood vessel diameter, the center of a heartbeat, and the heartbeat end position, may be used.

As also illustrated in FIG. 6C, during a period from the diastole d to the systole s of the heart, the vascular wall moves a lot (the moving velocity of the vascular wall is high). For this reason, the B-mode image during this period does not have high image quality compared to other positions. When the tracking of the vascular wall described below is performed, if a location where the vascular wall moves a lot is set as the start point, tracking errors are likely to occur.

For this reason, it is preferable that the predetermined position is set outside a period after the diastole d of the heart, that is, the heartbeat start position and before the systole s.

As also illustrated in FIG. 6C, before the diastole of the heart, that is, before the heartbeat start position, the movement of the vascular wall is gentle, and a high-quality B-mode image is obtained. When measurement using the displacement of the vascular wall, such as measurement of the vascular wall elasticity, is performed, in many cases, the period from the diastole to the systole of the heart in which the blood vessel diameter is changed from minimum to maximum is important for analysis.

Accordingly, it is preferable that a predetermined position where the B-mode image 64 is displayed is particularly set to the period from the heartbeat start position to the early 20% of the heartbeat. That is, it is preferable that the predetermined position is set to the period of 0.2 t before the heartbeat (including the heartbeat start position) illustrated in FIG. 6C.

In the ultrasound diagnostic apparatus 10 of the invention, a predetermined position where the B-mode image 64 is displayed may be set fixedly in advance as default or the operator, such as a physician, may select and set a predetermined position.

When the operator selects and sets a predetermined position, a plurality of positions, such as “the position corresponding to 10% before the heartbeat starts”, “the heartbeat start position”, “the position of the systole of the heart”, “the center of the heartbeat”, and “the heartbeat end position” may be set as options, such that the operator may select and set a predetermined position. Alternatively, an arbitrary position may be selected during a period (time axis) from the early 20% of the heartbeat to the late 20% of the heartbeat and may be set as a predetermined position. Alternatively, the method of selecting one of the set options and the method of selecting an arbitrary position on the time axis may be selected.

In the invention, the predetermined position is not limited to one, and two or more predetermined positions may be set.

For example, as the method of simply measuring the elastic modulus of the vascular wall, a method which calculates the ratio (Dd/Ds) of the minimum diameter (Dd) to the maximum diameter (Ds) of the blood vessel in one heartbeat is known. In response to this situation, the heart diastole (heartbeat start position) in which the blood vessel has the minimum diameter and the heart systole in which the blood vessel has the maximum diameter may be set as the predetermined positions.

When a plurality of predetermined positions are set as described above, a plurality of B-mode images may be switched and displayed. Alternatively, when the display 18 has a sufficient display space, a plurality of B-mode images may be displayed simultaneously along with an M-mode image.

If the lines of the heartbeats is displayed in the M-mode image 65, and the B-mode image 64 becomes the image of the predetermined position (as described above, in the illustrated example, the image of the heartbeat start position) corresponding to the selected heartbeat, the selection line 62 in the B-mode image becomes a solid line and is movable in the left-right direction by the trackball. That is, the selection line 62 is in the active state. Simultaneously, all the lines representing the heartbeats of the M-mode image become a broken line. Whether or not various lines are active may be distinguished by changing the line color instead of or in addition to the line type in a similar manner as described above.

In this state, if the selection line 62 is moved in the left-right direction by the trackball, the display processor 52 reads an M-mode image corresponding to the position of the selection line 62 from the storage unit 36, and displays the image on the display 18 along with the detection result of the heartbeats. That is, the selection line 62 is moved by the trackball even after freeze, thereby selecting the display position (display line) of the M-mode image 65 in the B-mode image 64 over the entire region in the azimuth direction in the B-mode image 64.

Therefore, according to this example, the M-mode image 65 of an arbitrary position in the azimuth direction of the set ROI 60 is displayed, such that the M-mode image 65 and an image corresponding to each heartbeat in the M-mode image can be observed and confirmed.

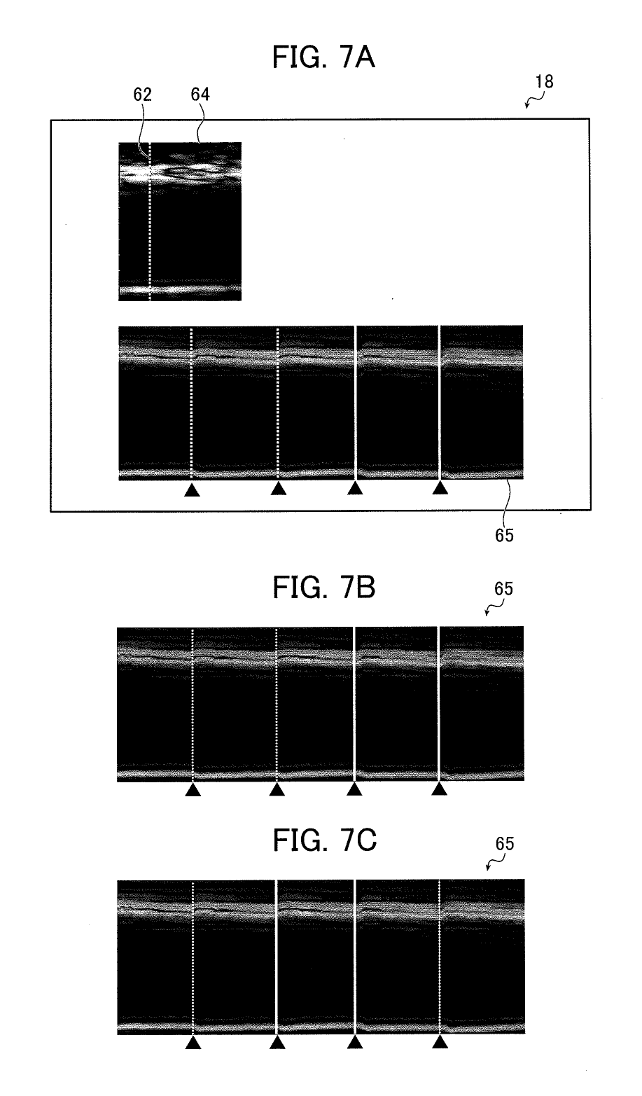

If the set button is depressed in a state where the selection line 62 of the B-mode image 64 is movable, it is determined that the display position (display line) of the M-mode image is selected. As illustrated in FIG. 7A, the selection line 62 of the B-mode image 64 becomes a broken line, such that the movement by the trackball is impossible. Simultaneously, lines indicating the latest heartbeat and automatically selected by the heartbeat selector 48 become a solid line in the M-mode image 65.

If the lines indicating the latest heartbeat become a solid line in the M-mode image 65, the heartbeat is selectable by the trackball.

When the set button is depressed, as described above, the latest heartbeat is selected (automatically selected by the heartbeat selector 48). In response to this, in the display of the M-mode image 65, as illustrated in FIGS. 7A and 7B, the lines indicating the latest heartbeat become solid lines and the heartbeat is selected.

In this state, for example, if the trackball rotates left, a signal of the rotation is supplied to the heartbeat selector 48. The heartbeat selector 48 determines that the second latest heartbeat is selected in accordance with the rotation, and supplies information indicating this effect to the display processor 52. The display processor 52 performs a process in accordance with the information such that, as illustrate in FIG. 7C, a line corresponding to the end of the latest heartbeat becomes a broken line, lines corresponding to a new heartbeat become solid lines, and the heartbeat is selected.

If the trackball further rotates left, the heartbeat selector 48 determines that the third latest heartbeat is selected in accordance with the rotation, and supplies information indicating this effect to the display processor 52. When this happens, a line corresponding to the end of the second latest heartbeat becomes a broken line, and lines corresponding to the third latest heartbeat become solid lines and the heartbeat is selected.

If the trackball rotates right, similarly, lines corresponding to later heartbeats are sequentially selected.

When the selected heartbeat changes with the movement of the trackball, the display processor 52 reads a B-mode image corresponding to the predetermined position of the selected heartbeat in accordance with information relating to the selected heartbeat from the storage unit 36 and displays the B-mode image on the display 18. That is, the B-mode image 64 is changed to the image of the predetermined position of the newly selected heartbeat.

In the illustrated example, the heartbeat start position is set as the predetermined position. Accordingly, for example, as illustrated in FIG. 7C, when the second latest heartbeat is selected by the trackball, the display processor 52 reads a B-mode image of the start position (start time) of the heartbeat from the storage unit 36 and displays the B-mode image on the display 18 as the B-mode image 64.

If the set button is depressed in a state where the heartbeats are selectable, it is determined that the selection of the heartbeats ends, the selected heartbeat is confirmed, and a state where fine adjustment of the selected heartbeat can be performed is reached.

If a heartbeat in the M-mode image 65 displayed on the display 18 is selected and confirmed, the heartbeat selector 48 changes information relating to the selected heartbeat in all the M-mode images stored in the storage unit 36 (that is, the M-mode images over the entire region in the azimuth direction of the B-mode image 64) as necessary. That is, in the illustrated example, when a heartbeat other than the latest heartbeat is selected and confirmed, information relating to the selected heartbeat is changed to the newly selected and confirmed heartbeat.

If the selected heartbeat is confirmed, information relating to the selected heartbeat is supplied to the tracker 42.

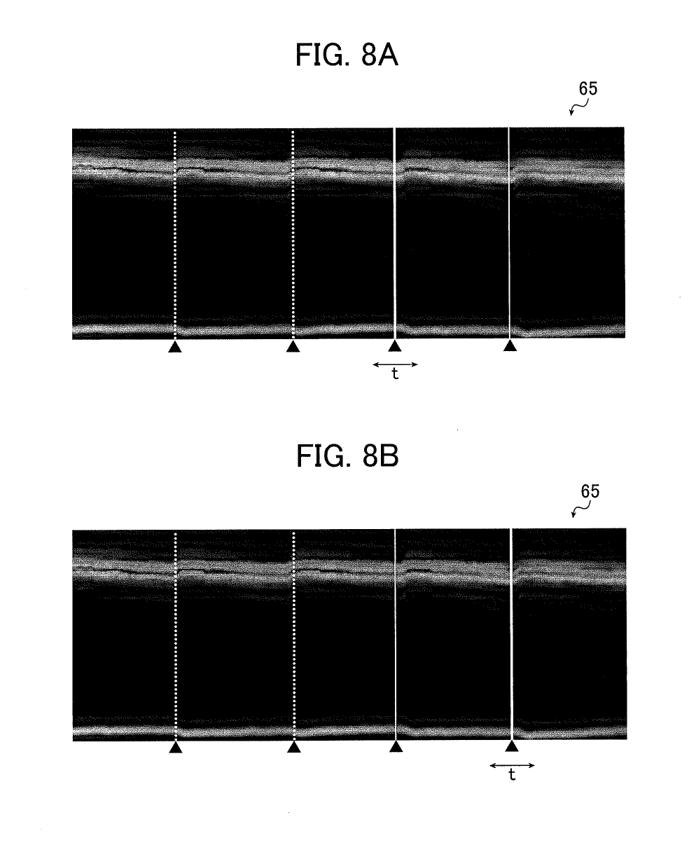

As an example, if it is determined that the last heartbeat is selected (that is, there is no change from the heartbeat automatically selected) and the set button is depressed, as illustrated in FIG. 8A, first, a line corresponding to the end of the selected heartbeat becomes a thin line, and the position (time) of a line corresponding to the start of the selected heartbeat is movable in the left-right direction (time direction) by the trackball as indicated by an arrow t, such that fine adjustment of the start position of the heartbeat can be performed.

If the set button is depressed after the start position of the heartbeat is adjusted by the trackball as required, as illustrated in FIG. 8B, a line corresponding to the end of the selected heartbeat becomes a normal solid line, and a line corresponding to the start of the selected heartbeat becomes a thin line. Accordingly, the position of the line corresponding to the end of the selected heartbeat is movable in the left-right direction by the trackball as indicated by the arrow t, such that fine adjustment of the end position of the heartbeat can be performed.

Although the result of fine adjustment of the heartbeat may be reflected only in the M-mode image 65 subjected to fine adjustment, it is preferable that the result is also reflected in all the M-mode images stored in the storage unit 36.

When the start position of the heartbeat is adjusted, the display processor 52 reads the B-mode image at the adjusted heartbeat start position from the storage unit 36, and the B-mode image 64 displayed on the display 18 is changed to this image.

The result of heartbeat fine adjustment is also supplied to the display processor 52. When the predetermined position in the selected heartbeat varies with fine adjustment of the heartbeat, the display processor 52 reads a B-mode image according to the varied predetermined position from the storage unit 36 and displays the B-mode image on the display 18.

In the illustrated example, the heartbeat start position is set as the predetermined position. Accordingly, after the heartbeat start position is adjusted as necessary, the display processor 52 reads a B-mode image corresponding to the adjusted heartbeat start position from the storage unit 36 and displays the B-mode image on the display 18.

When the result of fine adjustment of the heartbeat is also reflected in all the M-mode images stored in the storage unit 36, it is preferable that variation of the predetermined position according to fine adjustment of the heartbeat is reflected in all the M-mode images stored in the storage unit 36.

If the set button is depressed in a state where the end position of the selected heartbeat is adjustable, the state where the selection line 62 of the B-mode image 64 illustrated in FIG. 6A is movable, that is, the state where the display line of the M-mode image 65 is selectable in the B-mode image 64 is returned.

That is, in the ultrasound diagnostic apparatus 10 of the illustrated example, the processes “display line selection” →>“heartbeat selection” → “heartbeat fine adjustment” can be repeatedly performed. In other words, the processes “display line selection” → “heartbeat selection” → “heartbeat fine adjustment” may be performed in a looped manner.

Accordingly, it becomes possible to more suitably select the heartbeat most appropriate for analysis to measure the vascular wall elasticity described below from all the stored M-mode images.

If the “AW Det” button of the touch panel, not the set button, is depressed in a state where the position corresponding to the end of the selected heartbeat is adjustable, as illustrated in FIG. 9, the selection line 62 of the B-mode image 64 and the lines representing the heartbeats in the M-mode image 65 all become a broken line and are inoperable, and a vascular wall detection mode is reached.

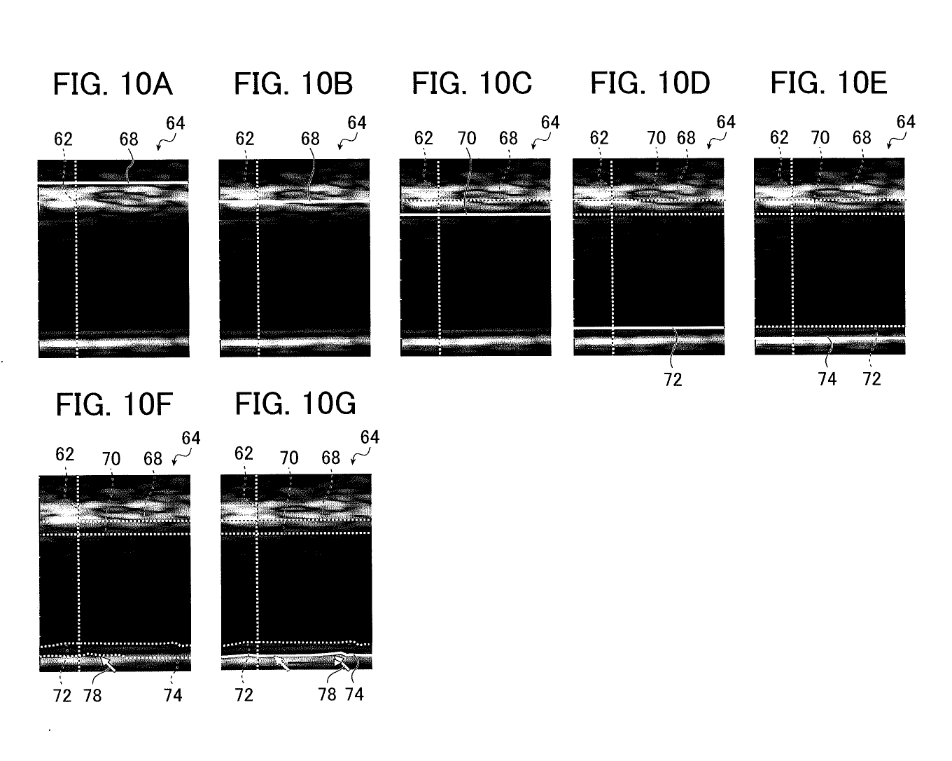

If the vascular wall detection mode is reached, first, as illustrated in FIG. 10A, a line 68 corresponding to the adventitia-media boundary of the blood vessel anterior wall is displayed in the B-mode image 64.

The line 68 is parallel-movable in the up-down direction (depth direction) by the trackball. As illustrated in FIG. 10B, after the line 68 is moved to the position of the adventitia-media boundary of the blood vessel anterior wall by the trackball, the set button is depressed.

If the set button is depressed, as illustrated in FIG. 10C, the line 68 corresponding to the adventitia-media boundary of the blood vessel anterior wall becomes a broken line and is confirmed in the B-mode image 64, and a line 70 corresponding to the intima-lumen boundary of the blood vessel anterior wall is displayed.

Similarly, the line 70 is movable in the up-down direction by the trackball, and after the line 70 is moved to the position of the intima-lumen boundary of the blood vessel anterior wall, the set button is depressed.

If the set button is depressed in a state where the line 70 is parallel-movable, as illustrated in FIG. 10D, the line 70 corresponding to the intima-lumen boundary of the blood vessel anterior wall becomes a broken line and is confirmed in the B-mode image 64, and a line 72 corresponding to the intima-lumen boundary of the blood vessel posterior wall is displayed. Similarly, after the line 72 is moved to the position of the intima-lumen boundary of the blood vessel posterior wall by the trackball, the set button is depressed.

If the set button is depressed in a state where the line 72 is movable, as illustrated in FIG. 10E, the line 72 corresponding to the intima-lumen boundary of the blood vessel posterior wall becomes a broken line and is confirmed in the B-mode image 64, and a line 74 corresponding to the adventitia-media boundary of the blood vessel posterior wall is displayed. Similarly, after the line 74 is moved to the position of the adventitia-media boundary of the blood vessel posterior wall by the trackball, the set button is depressed.

The information of each boundary of the vascular wall is supplied to the boundary detector 40.

If the set button is depressed in a state where the line 74 is movable, the setting of the lines corresponding to all the boundaries ends, and the boundary detector 40 automatically detects the intima-lumen boundary and the adventitia-media boundary of the posterior wall using the set line 72 of the intima-lumen boundary and the set line 74 of the adventitia-media boundary. The result of the automatic detection of both boundaries is sent to the display processor 52 and the tracker 42, and as illustrated in FIG. 10F, the detection result is displayed.

The method of automatically detecting these boundaries is not particularly limited, and various methods may be used. As an example, a method is used in which a B-mode image is analyzed, continuous high-luminance portions at the positions of the line 72 and the line 74 are tracked to detect the intima-lumen boundary and the adventitia-media boundary.

If the automatic detection of the intima-lumen boundary and the adventitia-media boundary of the blood vessel posterior wall by the boundary detector 40 ends, as illustrated in FIG. 10F, a cursor 78 is displayed in the B-mode image 64 (the cursor 78 is not displayed until the automatic detection of the blood vessel posterior wall ends).

The cursor 78 is movable by the trackball. If the cursor 78 is moved to the line representing the automatically detected intima-lumen boundary or adventitia-media boundary, and the set button is depressed, the line closer to the cursor 78 becomes a solid line. The line which has become a solid line is correctable.

For example, as illustrated in FIG. 10G, it is assumed that the line 74 representing the adventitia-media boundary is selected and becomes a solid line. If the cursor 78 is moved along the line 74 by the trackball, and the set button is depressed again, the line 74 of the region tracked by the cursor is detected again by the boundary detector 40 and rewritten, and the result is sent to the tracker 42.

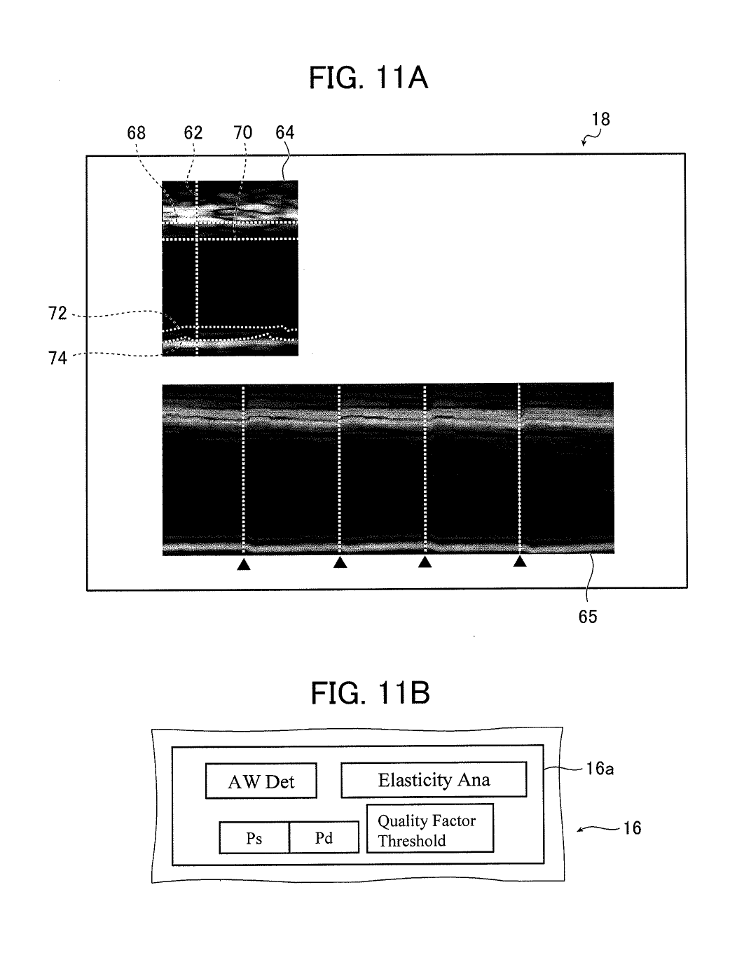

If the automatic detection of the intima-lumen boundary and the adventitia-media boundary of the posterior wall ends, and if necessary, the blood vessel posterior wall is corrected, as illustrated in FIG. 11A, a state where all lines become a broken line is reached, and as illustrated in FIG. 11B, the “Elasticity Ana” button of the touch panel 16a is selectable.

After the “Elasticity Ana” button is selectable, the blood pressure in the heart systole of the subject is input by the “Ps” button, the blood pressure in the heart end diastole of the subject is input using the “Pd” button, and the reliability threshold value is input using the “Quality Factor Threshold” button. These numerical values may be input by a known method.

The input of the blood pressure of the subject and the reliability threshold value is not limited to the input after the detection of the vascular wall boundaries has ended. The input may be performed at any timing before analysis described below starts (before the “Elasticity Ana” button described below is depressed).

In the ultrasound diagnostic apparatus 10, it is usual that before a diagnosis is performed, the subject information is acquired or input. Accordingly, when the subject information includes the information of the blood pressure, the information of the blood pressure may be used.

If the blood pressure of the subject and the reliability threshold value are input, and the “Elasticity Ana” button is depressed, analysis of the B-mode image starts, and the elastic modulus of the vascular wall is calculated.

If the “Elasticity Ana” button is depressed, first, the tracker 42 tracks the motions of the blood vessel anterior wall (adventitia-media boundary and intima-lumen boundary) and the blood vessel posterior wall (intima-lumen boundary and adventitia-media boundary) in the selected heartbeat in the M-mode image 65. That is, the blood vessel anterior wall and posterior wall are tracked.

The tracking of the vascular wall in the M-mode image 65 is performed with the adventitia-media boundary of the blood vessel anterior wall, the intima-lumen boundary of the blood vessel anterior wall, the intima-lumen boundary of the blood vessel posterior wall, and the adventitia-media boundary of the blood vessel posterior wall previously detected (set) in the B-mode image 64 as a positional starting point (a starting point in the focal depth direction).

Meanwhile, a temporal starting point for the tracking of the vascular wall in the M-mode image 65 is the position (time phase) corresponding to the B-mode image 64 of the predetermined position of the selected heartbeat displayed on the display 18. That is, the time at which the B-mode image 64 is captured in the M-mode image 65 becomes the starting point of the tracking. Therefore, in the illustrated example, the start position of the selected heartbeat becomes the starting point of the tracking.

As described above, the B-mode image 64 which specifies the vascular wall boundaries is the B-mode image which corresponds to the predetermined position of the selected heartbeat set during the period from the early 20% of the heartbeat to the late 20% of the heartbeat.

Accordingly, in the illustrated example, since the B-mode image 64 which becomes the starting point for the tracking corresponds to the selected heartbeat or is close to the selected heartbeat, it is possible to perform the tracking of the vascular wall in the M-mode image 65 in the selected heartbeat without being affected by tracking errors, extra noise, or the like.

In the ultrasound diagnostic apparatus 10, as a preferred form, not only the detected (set) boundaries of the vascular wall but also one or more measurement points in the depth direction may be set in the blood vessel posterior wall. In this way, when one or more measurement points are set in the blood vessel posterior wall, the tracking of the vascular wall is performed at each measurement point.

The measurement point in the vascular wall may be set in advance, may be automatically set on the basis of a specific algorithm, or may be set by the operator of the ultrasound diagnostic apparatus 10 while viewing the image. These may be used in combination.

The method of tracking the vascular wall in the M-mode image 65 is not particularly limited, and there are a method which uses continuity of images (luminance) from the starting point of the tracking, a pattern matching method, a zero crossing method, a tissue Doppler method, phase difference tracking, and the like. Of these, any method may be used.

The tracking result of the vascular wall in the M-mode image by the tracker 42 is supplied to the elastic modulus calculator 50 and the display processor 52.

The elastic modulus calculator 50 first produces the change waveform of the thickness of the vascular wall (intima-media) and the change waveform of the blood vessel diameter (inner diameter) from the tracking result of the vascular wall. As described above, when one or more measurement points are set in the vascular wall, the change waveform of the vascular wall is produced between the measurement points.

The change waveform of the thickness of the vascular wall and the change waveform of the blood vessel diameter are sent to the display processor 52.

The elastic modulus calculator 50 calculates strain in the radial direction of the blood vessel using Equation (1).

εᵢ=Δhᵢ/h_{di} . . . (1)

In Equation (1), εᵢ denotes strain in the radial direction of the blood vessel between the measurement points, Δhᵢ denotes the maximum value of a change in thickness of the vascular wall between the measurement points in the heart systole in which the vascular wall is smallest in thickness in one heartbeat, and h_{di} denotes the thickness between the measurement points in the heart end diastole in which the vascular wall is largest in thickness.

The elastic modulus calculator 50 calculates an elastic modulus E_{θi} in the circumferential direction of the vascular wall by Equation (2) using the maximum value and the minimum value of the blood pressured input in advance.

E_{θi}=½* [1+(r_{d}/h_{d})]*[Δp/(Δhᵢ/h_{di})] . . . (2)

An elastic modulus Eᵣᵢ in the radial direction of the vascular wall may be calculated by Equation (3).

Eᵣᵢ=Δp/(Δhᵢ/h_{di}) . . . (3)

In Equations (2) and (3), Δhᵢ and h_{di} are the same as described above, Δp denotes a blood pressure difference between the heart systole and the heart end diastole, r_{d} denotes the radius of the vascular lumen in the heart end diastole, and h_{d} denotes the thickness of the vascular wall in the heart end diastole.

After the elastic modulus is calculated, the elastic modulus calculator 50 calculates reliability of the elastic modulus.

The method of calculating reliability of the elastic modulus is not particularly limited, and various known methods may be used. As an example, there is a method in which the waveforms of changes in the blood vessel diameter by the heartbeats of many people, such as 1000 persons are prepared, the model waveform of the change in the blood vessel diameter is created from many waveforms, and reliability of the calculated elastic modulus is calculated using the amount of a shift from the model waveform.

As described above, if a heartbeat is selected and confirmed in the M-mode image displayed on the display 18, the same heartbeat is selected in all the M-mode images stored in the storage unit 36.

Accordingly, the processes, such as the tracking of the vascular wall, the production of the change waveforms of the thickness of the vascular wall and the blood vessel diameter, the calculation of strain of the vascular wall, and the calculation of the elastic modulus of the vascular wall and reliability of the elastic modulus, are performed in the selected heartbeat for not only the M-mode image 65 displayed on the display 18 but also all the M-mode images stored in the storage unit 36. That is, the processes, such as calculation of the elastic modulus of the vascular wall, in the selected heartbeat are performed over the entire region in the azimuth direction of the B-mode image 64 displayed on the display 18 using the corresponding M-mode image.

These results are added to the M-mode images stored in the storage unit 36 as information.

After the calculation over the entire region in the azimuth direction ends, the elastic modulus calculator 50 calculates the average value (E_{θave}) of the elastic modulus of the vascular wall, the average value (Strₐᵥₑ) of strain of the vascular wall, and the average value (QFₐᵥₑ) of reliability of the elastic modulus.

If the calculation ends, the result is displayed on the display 18.

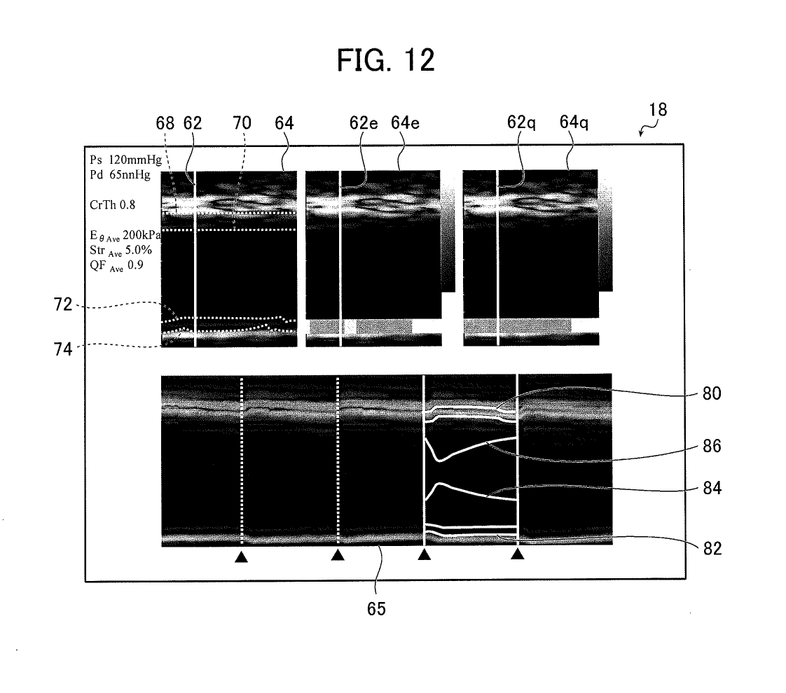

FIG. 12 illustrates an example. In the illustrate example, on the right side of the displayed B-mode image 64, the elastic modulus of the blood vessel posterior wall represented in the B-mode image 64 is displayed by a B-mode image 64e. On the right side of the B-mode image 64e which displays the elastic modulus of the blood vessel posterior wall, reliability of the elastic modulus of the vascular wall is displayed by a B-mode image 64q in a similar manner.

On the left side of the B-mode image 64, the average value (E_{θave}) of the elastic modulus of the vascular wall, the average value (Strₐᵥₑ) of strain of the vascular wall, and the average value (QFₐᵥₑ) of reliability of the elastic modulus are respectively displayed.

The elastic modulus of the vascular wall is displayed in a strip shape in the B-mode image 64e to overlap the blood vessel posterior wall automatically detected (and corrected as necessary) in the B-mode image 64. On an upper right side of the B-mode image 64e, the index of the elastic modulus is displayed. In the illustrated example, the higher the image density, the higher the elastic modulus.

That is, in the B-mode image 64e, the density of the strip overlapping the blood vessel posterior wall represents the elastic modulus of the vascular wall at the corresponding position of the blood vessel.

Similarly, reliability of the elastic modulus is displayed in a strip shape in the B-mode image 64q to overlap the blood vessel posterior wall automatically detected in the B-mode image 64. On an upper right side of the B-mode image 64q, the index of reliability of the elastic modulus is displayed. In the illustrated example, the higher the image density, the higher reliability of the elastic modulus.

That is, in the B-mode image 64q, the density of the strip overlapping the blood vessel posterior wall represents reliability of the vascular wall elastic modulus at the corresponding position of the blood vessel.

The magnitude of the elastic modulus or reliability of the elastic modulus may be realized by changing the image color instead of or in addition to the image density.

In the display of the result illustrated in FIG. 12, the result is automatically omitted at the position in the azimuth direction where reliability of the result is lower than a threshold value input in advance.

With regard to the position where the result is omitted, as represented in a right corner portion of the result display of the elastic modulus in the B-mode image 64e or a right corner portion of the result display of reliability in the B-mode image 64q, the display of the strip is thinned.

In the lower M-mode image 65, a tracking result 80 of the blood vessel anterior wall, a tracking result 82 of the blood vessel posterior wall, a change waveform 84 of the blood vessel diameter, and a change waveform 86 of the thickness of the vascular wall in the M-mode image are displayed in the selected heartbeat.

As described above, when one or more measurement points are set in the vascular wall in the depth direction, the change waveform of the blood vessel thickness may be output between the measurement points.

If the measurement result of the elastic modulus of the vascular wall or the like is displayed on the display 18, the selection line 62 becomes a solid line in the B-mode image 64, and is movable in the azimuth direction by the trackball.

If the selection line 62 is moved in the B-mode image 64, the display processor 52 reads the M-mode image corresponding to the position of the selection line 62 from the storage unit 36 and displays the M-mode image on the display 18. That is, if the selection line 62 is moved by the trackball, the M-mode image 65 is changed to the M-mode image at the position of the selection line 62, and the tracking results 80 and 82 of the blood vessel anterior wall and the blood vessel posterior wall, the change waveform 84 of the blood vessel diameter and the change waveform 86 of the thickness of the vascular wall in the M-mode image are changed to data at the position of the selection line 62 of the B-mode image 64.

Accordingly, it is possible to select the display line for displaying the M-mode image 65 and the analysis result over the entire region in the azimuth direction of the B-mode image.

A selection line 62e of the B-mode image 64e and a selection line 62q of the B-mode image 64q also move in synchronization with the movement of the selection line 62 in the B-mode image 64.

After the set button is depressed, in the B-mode image 64e and the B-mode image 64q, if the selection line 62e and the selection line 62q are moved by the trackball to select an arbitrary region in the azimuth direction, and thereafter, the set button is depressed again, the selected region is handled in a similar manner to the above-mentioned region where reliability is lower than the threshold value, and data is deleted.

That is, a tester views the result, and when there is a location where the waveform or the like seems to be extraordinary, data can be deleted, thereby making it possible to perform more accurate analysis.

The state after the deletion of data may be returned in a previous state by depressing a Delete button or the like.

Although the ultrasound diagnostic apparatus of the invention has been described in detail, the invention is not limited to the foregoing examples, and various modifications or improvements may be of course made without departing from the scope of the invention.

The ultrasound diagnostic apparatus of the invention can be suitably used in medical practice for the diagnosis of arteriosclerosis which causes myocardial infarction, angina pectoris, brain diseases, and the like.

What is claimed is:

1. An ultrasound diagnostic apparatus comprising:
an ultrasound probe which has ultrasound transducers transmitting ultrasonic waves, receiving an ultrasonic echo reflected by a subject, and outputting a reception signal according to the ultrasonic echo received;
an image producer which produces a B-mode image and an M-mode image from the reception signal output from the ultrasound transducers;
a display unit;
a display processor which displays at least one of the B-mode image and the M-mode image produced by the image producer on the display unit;
a freezer which freezes a display image to a still image in a state where the B-mode image and the M-mode image are displayed; and
a heartbeat selector which selects a heartbeat in the M-mode image in a state where the B-mode image and the M-mode image are frozen as still images,
wherein, after the heartbeat is selected in the M-mode image, in correspondence to a predetermined position set in advance within a period from an early 20% of the heartbeat to a late 20% of the heartbeat relative to a length of the heartbeat, the display processor displays a B-mode image of the predetermined position in the selected heartbeat on the display unit.

2. The ultrasound diagnostic apparatus according to claim 1, further comprising:
a heartbeat detector which detects heartbeats in the M-mode image,
wherein, after the heartbeat detector detects the heartbeats, the display processor displays all the heartbeats detected by the heartbeat detector in the M-mode image.

3. The ultrasound diagnostic apparatus according to claim 2, wherein the heartbeat detector analyzes the M-mode image to detect the heartbeats in the M-mode image.

4. The ultrasound diagnostic apparatus according to claim 1, further comprising:
a selector which selects the predetermined position.

5. The ultrasound diagnostic apparatus according to claim 2,
wherein the heartbeat selector decides that a latest heartbeat wholly acquired from start to end in an M-mode image is selected among the heartbeats detected by the heartbeat detector.

6. The ultrasound diagnostic apparatus according to claim 1,
wherein the predetermined position is set outside a period after a heart diastole and before a heart systole.

7. The ultrasound diagnostic apparatus according to claim 1, further comprising:
a position adjuster which adjusts a position of the heartbeat selected by the heartbeat selector.

8. The ultrasound diagnostic apparatus according to claim 7,
wherein, after the position of the heartbeat is adjusted by the position adjuster, the display processor changes the B-mode image to be displayed on the display unit to an image of a corresponding position in accordance with heartbeat position adjustment and the predetermined position.

9. The ultrasound diagnostic apparatus according to claim 1, further comprising:
a vascular wall boundary setter which sets a position of a vascular wall boundary in the B-mode image.

10. The ultrasound diagnostic apparatus according to claim 1, further comprising:
a region-of-interest setter which sets a region of interest in the B-mode image displayed on the display unit.

11. The ultrasound diagnostic apparatus according to claim 10,
wherein a frame rate of ultrasonic waves by the ultrasound transducers is increased in response to an instruction to set the region of interest to be higher than before the instruction to set the region of interest.
